(19) United States
(12) Patent Application Publication
(10) Pub. No.: US 2007/0129447 A1
(43) Pub. Date: Jun. 7, 2007

(54) Method of drug therapy in treatment of cardiac arrhythmias

(76) Inventor: Jasbir S. Sra, Pewaukee, WI (US)

Correspondence Address: JANSSON SHUPE & MUNGER LTD., 245 MAIN STREET, RACINE, WI, 53403, US

(21) Appl. No.: 11/606,498

(22) Filed: Nov. 30, 2006

Related U.S. Application Data
(60) Provisional application No. 60/741,978, filed on Dec. 2, 2005.

(51) Int. Cl. A61K 31/138

(52) U.S. Cl. 514/651

(57) ABSTRACT

A method for treatment of cardiac arrhythmias is disclosed. The method comprises the combining of a sodium channel blocking medication and a potassium channel blocking medication into a therapeutic dosage and the delivery of the therapeutic dosage to a patient in treatment of a heart rhythm problem. The heart rhythm problems being treated include atrial fibrillation and atrial or ventricular tachycardia. Preferably, the therapeutic dosage is at least one tablet and the concentration of each medication is determined based upon the heart problem being treated, the underlying heart rate, the QT interval and other comorbid conditions of the patient. A pharmaceutical composition for the therapeutic treatment of cardiac arrhythmias in a patient is also provided having a combination of a sodium channel blocking medication and a potassium channel blocking medication. Under certain conditions of the patient, the composition can also include a beta blocking medication.

### RELATED APPLICATION

This application claims the benefit of U.S. Provisional Application No. 60/741,978 filed on Dec. 2, 2005, the contents of which is incorporated by reference herein in its entirety.

### FIELD OF THE INVENTION

This invention is related generally to the treatment of heart rhythm problems and, more particularly, to a method for treating cardiac arrhythmias using anti-arrhythmic medications.

### BACKGROUND OF THE INVENTION

Heart rhythm problems or cardiac arrhythmias are extremely common heart problems and a major cause of mortality and morbidity. The mechanisms of these rhythm problems involve aberrant electrical circuits moving in precise anatomically determined paths. To cure these rhythm problems, therefore, it would be logical to block these circuits as precisely as possible with little or no side effects.

Examples of rhythm problems often encountered in clinical practice include atrial fibrillation (AF), cardiac arrest or sudden cardiac death (SCD) due to ventricular tachycardia/ventricular fibrillation (VT/NVF), atrial flutter, and other forms of atrial and ventricular arrhythmia. During the past 20 years, cardiac electrophysiology has evolved into a clinical tool to diagnose these cardiac arrhythmias. However, essential to the management of any particular cardiac rhythm problem is a thorough understanding of the mechanism of its initiation and sustenance. A short discussion of these arrhythmias and their mechanisms is thus useful in understanding the complexity of the problem and how this invention helps facilitate the treatment of these arrhythmias.

Atrial fibrillation is dysrhythmia of the atria or the upper chambers of the heart in which the atria stop contracting and begin to fibrillate or quiver. Atrial fibrillation is the most commonly sustained arrhythmia encountered in clinical practice and, as recent data suggests, the most common arrhythmia-related cause of hospital admissions. Estimates indicate that 2.2 million people in the United States alone have AF and that 160,000 new cases are diagnosed every year. Patients with AF have a higher incidence of further complications such as stroke and heart failure and bear the ominous prognosis of increased overall mortality.

Recently it has been shown that premature atrial contractions can act as triggers that initiate paroxysms of AF. These premature ectopic beats have been shown to originate predominantly in the pulmonary veins. The inability to reproducibly identify the precise location of these trigger sites of AF, however, limits their ablation with a catheter. Despite the infrequent and nonreproducible nature of premature atrial contractions limiting the utility of trigger site ablation, a variety of surgical and nonsurgical catheter ablation techniques have been used to isolate the pulmonary veins from the left atrium because of the critical role of the pulmonary veins in the generation of AF.

Intra-operative complete isolation of the pulmonary veins using various energy sources in patients undergoing open heart surgery has led to successful termination of AF in over 80% of patients. Trying to replicate this procedure nonsurgically, however, is lengthy and labor intensive. Usually two catheters are positioned inside the left atrium guided by fluoroscopy. As the left atrium-pulmonary vein junction cannot be seen, the catheters are swept around and only electrical signals are used to guide them to the locations where heat is delivered. As true anatomy is not visualized, the success rate of this procedure is low and only a limited number of patients qualify for this procedure.

For these reasons, anti-arrhythmic drugs still remain the first treatment of choice for these patients with AF. There are several types of anti-arrhythmic medications. Some work by blocking the inward sodium current while others block the outward potassium current. Potassium channel blockers have reverse use dependence. This means that they are more effective at slow heart rates. Sodium channel blockers, on the other hand, are more effective at fast heart rates.

Sudden cardiac death is defined as an unexpected natural death from an underlying cardiac cause within a short period of time. Most SCDs are caused by VT/VF. It is estimated that SCD accounts for approximately 300,000 cardiac deaths in the United States alone each year. SCD is the most common and, unfortunately, oftentimes the first manifestation of coronary artery disease. It is said to be responsible for approximately 50% of the deaths from cardiovascular disease in the United States.

The most commonly encountered form of VT typically originates in the vicinity of a healed myocardial infarction. The mechanism of VT is reentry associated with myocardial scarring. These reentrant circuits are, however, quite broad because of the nature of the scarring. Although implantable defibrillators are the treatment of choice for many of these patients, anti-arrhythmic medications are frequently chosen as an adjunct to such device therapy in order to treat either the VT/VF or the concurrent occurrence of AF in these patients due to the frequent shocks generated by the defibrillator.

Several other arrhythmias involving accessory connections between the atria and the ventricles such as atrial flutter, atrial tachycardia, and tachycardia are also extremely common. Each is a cause of significant morbidity and creates some risk of higher mortality. The mechanism of atrial flutter has fortunately now been identified. Ablation between the tricuspid annulus and inferior vena cava to directly form an anatomical barrier around the flutter circuit can successfully terminate further atrial flutter. Nevertheless, in a significant number of these patients, anti-arrhythmic drugs are still used to prevent recurrent arrhythmias.

Anti-arrhythmic drugs have, however, a serious side effect called Torsades de pointes. This is where a dangerous arrhythmia occurs in the lower chamber because of prolongation of the QT interval, the interval during which contraction of the ventricles occurs. In addition to this side effect, it has been found that only 50% to 60% of patients are even responsive to these medications.

An anti-arrhythmic medication that combines a drug having sodium channel blocking properties with a drug having potassium blocking properties would be highly desirable in improving the overall efficacy of these drugs in the treatment of refractory arrhythmias and in reducing their potential toxicity.

A primary object of the present invention is to provide an improved method in the treatment of cardiac arrhythmias that overcomes some of the problems and shortcomings of the prior art. Another object of this invention is to provide a novel method that combines and consolidates the properties of certain anti-arrhythmic drugs and uses them in different proportions to treat refractory heart rhythm problems.

### SUMMARY OF THE INVENTION

One aspect of this invention is directed to a method of treatment of cardiac arrhythmias that comprises the combining of a sodium channel blocking medication with a potassium channel blocking medication into a therapeutic dosage that is delivered to a patient in treatment of his heart rhythm problem. The therapeutic dosage is preferably in the form of at least one tablet that comprises an anti-arrhythmic drug combining both sodium and potassium channel block The therapeutic dosage can also be each of the sodium and potassium channel blocking medications in separate tablets that are then taken together by the patient.

In certain desirable embodiments of this invention, the heart rhythm problem being treated is atrial fibrillation. Certain preferred cases find the heart rhythm problem to be a rhythm problem of a heart chamber other than the left atrium. In such cases, it is most desirable that the heart rhythm problem be ventricular tachycardia, supraventricular tachycardia such as atrial flutter, atrial tachycardia or isolated premature atrial or ventricular beats.

Different concentrations of sodium and potassium channel blocking medications can be used. The concentration of each medication is preferably determined based upon the specific cardiac arrhythmia being treated, the underlying heart rate, the QT interval and other comorbid conditions of the patient. For example, a patient with a slow heart rate can be given medication dosage wherein the sodium channel blocking drug is at as high a concentration by weight as 75% while the potassium channel blocking drug is at 25%. A similar approach can be used with patients having a prolonged QT interval at baseline. On the contrary, patients who have a higher heart rate at baseline or who have high blood pressure at the onset can be given a dosage with a higher concentration by weight of the potassium channel blocker. In such cases, it is desirable that the dosage also include medication having some beta blocking effect.

Another aspect of this invention is directed to a pharmaceutical composition for the therapeutic treatment of a cardiac arrhythmia in a patient comprising a combination of a sodium channel blocking medication and a potassium channel blocking medication. In certain preferred embodiments, the combination is at least one tablet.

Most desirable is where the pharmaceutical composition or drug is for treatment of atrial fibrillation. Also preferred is where the composition's combination is for treatment of a rhythm problem of a heart chamber other than the left atrium. Highly desirable is where the sodium and potassium channel blocking medications each have a concentration and the concentration of each medication is determined based upon the cardiac arrhythmia being treated, underlying heart rate, QT interval and other comorbid conditions of the patient.

In another preferred embodiment of this drug, the sodium channel blocking medication is within a range of about 50 milligrams to about 400 milligrams. Also desirable is where the potassium channel blocking medication of the pharmaceutical composition is within a range of about 80 milligrams to about 400 milligrams. Most desirable is where the pharmaceutical composition also includes a beta blocking medication.

### BRIEF DESCRIPTION OF THE DRAWINGS

FIG. 1 is a flow diagram showing a process for treatment of cardiac arrhythmias in accordance with this invention.

FIG. 2 is a schematic illustration of the cardiac action potential showing different sites of drug action.

FIG. 3 depicts changes with an increase in the QT interval sufficient to trigger initiation of Torsades de pointes.

FIG. 4 is an example of a surface ECG showing a long QT interval.

FIG. 5 is an example of a surface ECG showing atrial fibrillation with a fast ventricular rate.

FIG. 6 is an example of a surface ECG showing a slow ventricular rate during atrial fibrillation.

### DETAILED DESCRIPTION OF PREFERRED EMBODIMENTS

While this invention is susceptible of embodiments in many different forms, there are shown in the attached drawings and in the detailed discussion that follows preferred embodiments in accordance with this invention.

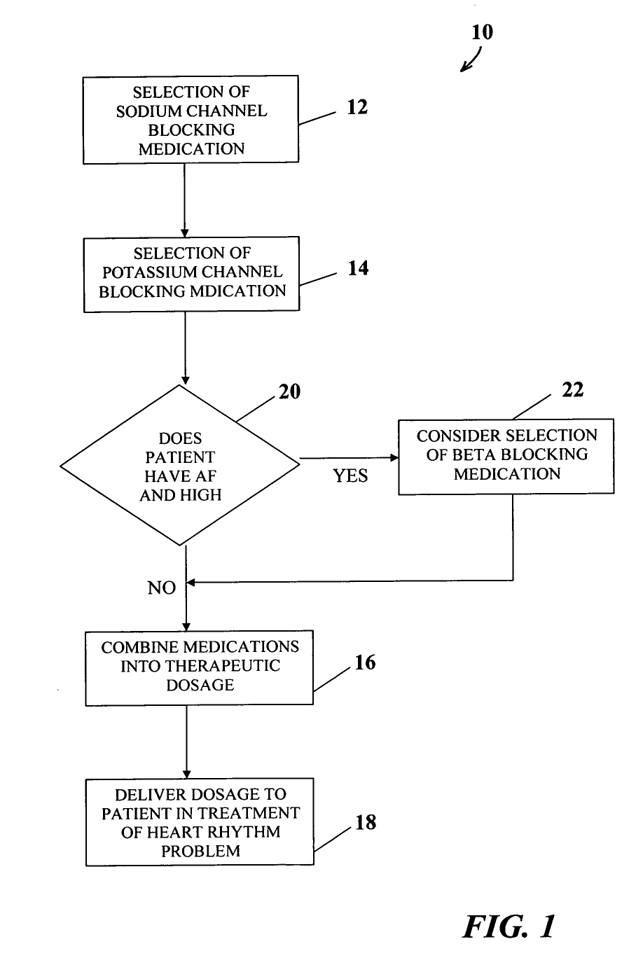

FIG. 1 shows a process 10 for treatment of cardiac arrhythmias according to the present invention. At step 12, a sodium channel blocking medication is selected. A heart beat involves both contraction (or depolarization) and relaxation (also called repolarization). Refractoriness of the heart muscle will determine how the heart tissue behaves in relationship to the electrical activity in the heart. Sodium channel blockade primarily delays conduction in the heart tissue and increases post repolarization refractoriness in a use dependent fashion. The therapeutic effect of this medication is therefore greater at higher heart rates.

At step 14, a potassium channel blocking medication is selected. Potassium channel blockade primarily delays repolarization and increases the refractory period of the heart muscle. This effect occurs in a reverse use dependent fashion, resulting in the maximum effect being seen during slower heart rates.

Step 16 in process 10 combines the medications selected into a single therapeutic dosage for the patient being treated, preferably a single tablet. At step 18, the dosage is delivered to the patient in treatment of a heart rhythm problem. This provides the patient with a drug having two different mechanisms of action. Combining the individual effects of sodium channel and potassium channel blockade in one drug dosage increases the refractory period over a wider range of heart rates. The synergistic effect of this combination of sodium and potassium channel blockades therefore achieves beneficial effects in the treatment of many different heart rhythms.

Depending upon the underlying heart rate, the QT interval and other comorbid conditions, the strength or concentration of the sodium and potassium channel blocking medications combined into the drug according to this invention will range from 50 milligrams to 400 milligrams for the sodium channel blocker and, correspondingly, from 400 milligrams to 80 milligrams for the potassium channel blocker.

In patients with an abnormal QT interval, such as one that exceeds, for example, 400 to 450 milliseconds, the concentration by weight of the potassium channel blocker in the therapeutic dosage formed will be at its lowest, preferably at 25% of the combination. Furthermore, at step 20, in patients who have high blood pressure along with faster heart rates due to atrial fibrillation, medication such as a beta blocker can be selected at step 22 to be combined with the sodium and potassium channel blockers to serve as a third component of the therapeutic dosage provided the patient in accordance with this invention.

One skilled in the art will recognize that the exact ordering of the steps as shown in FIG. 1 is not intended to be critical or limiting in the execution of process 10. In particular, step 14 may precede step 12 and another combination step 16 can precede the election at step 20.

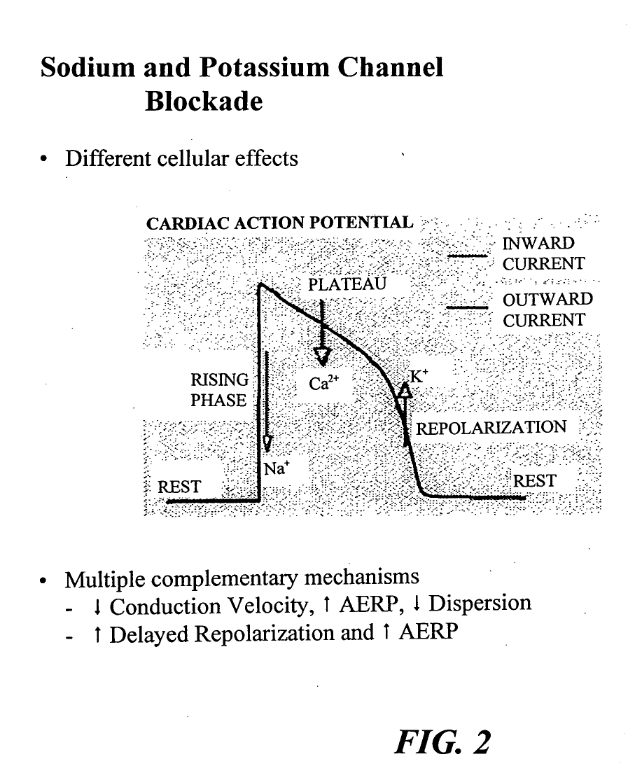

FIG. 2 demonstrates the action potential and how the combination of sodium and potassium channel blocking medications in accordance with this invention will have beneficial effects. The first part of the action potential is the rising phase. This phase is affected by the inward sodium current. The following repolarization phase is affected by the outward potassium current. Blocking the two simultaneously will have complimentary effects. Sodium channel blockade, which occurs at faster rates, will cause a decrease in conduction velocity, an increase in effective refractory period (ERP) and a decrease in dispersion. Potassium channel blockade, which predominantly occurs at slower heart rates, will cause a delayed repolarization and an increase in effective refractory period (ERP). Any changes in the QT interval due to the delayed repolarization by the potassium channel blocking medication will therefore be mitigated by the therapeutic impact of the sodium channel blocking medication in the combination dosage.

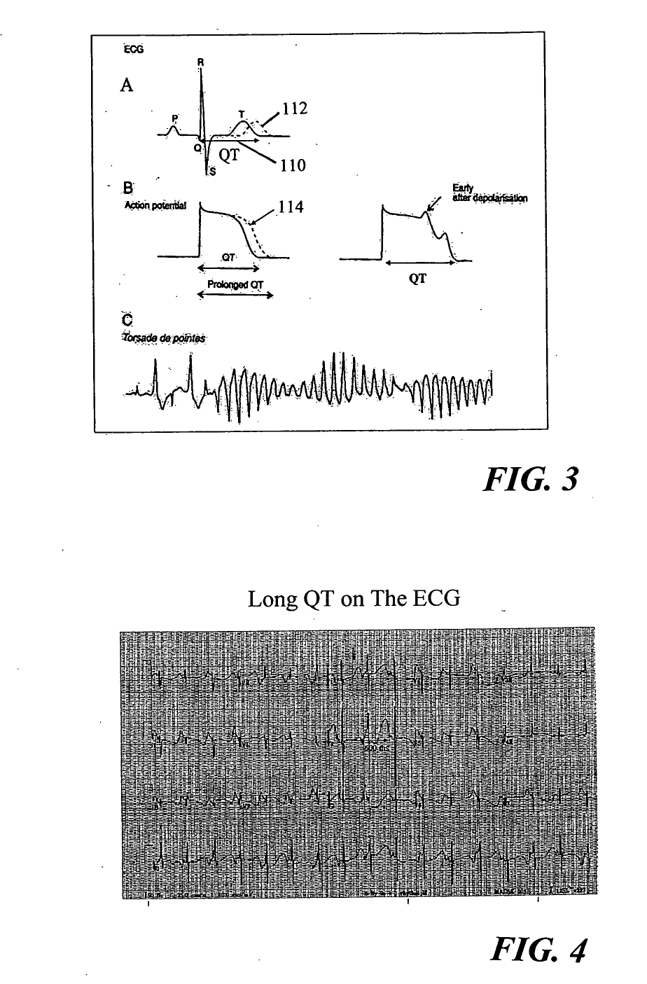

FIG. 3 depicts the changes seen upon an electrocardiogram (ECG) corresponding to the changes in the action potential due to treatment with only a potassium channel blockade. Panel A shows a sinus beat with the QT interval measurement shown by arrow 110. Panel B depicts the action potential itself. The action potential duration is depicted as the QT interval on the surface ECG. Change in the action potential duration leading to an increase in the QT interval is shown in Panels A and B by dashed lines 112 and 114, respectively. Panel C depicts the initiation of Torsades de pointes due to the long QT interval. The long QT is followed by the initiation of a ventricular tachycardia.

FIG. 4 depicts an ECG showing a long QT interval. The QT interval shown is 600 milliseconds while the normal interim is about 400 milliseconds.

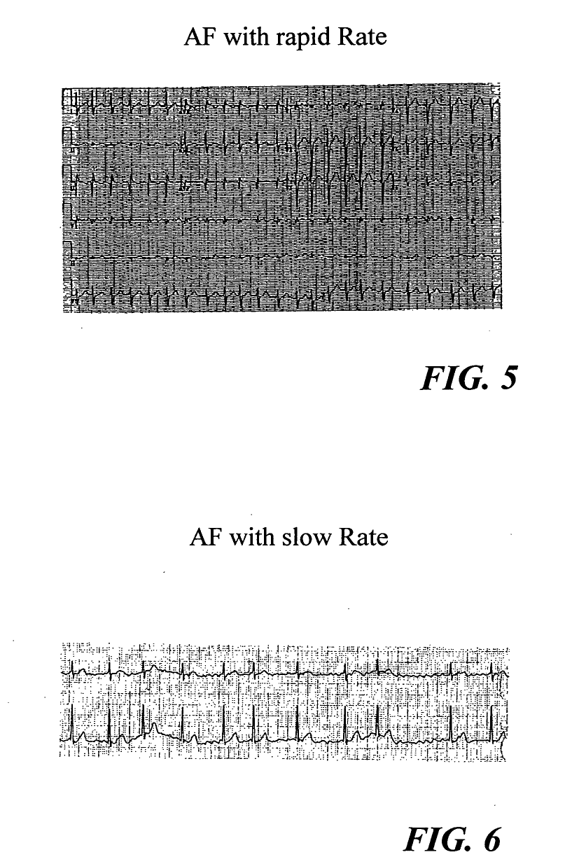

FIG. 5 depicts atrial fibrillation with a rapid ventricular rate. This type of arrhythmia is more likely to respond to a combination dosage where the potassium channel blockers and beta blockers together are at a higher concentration by weight than the sodium channel blockers. In contrast, FIG. 6 depicts where the ventricular rate is slower, a situation more likely to respond to a higher concentration of sodium channel blocking agents than the concentration of potassium channel blocking medication.

Although the invention has been described in conjunction with certain embodiments thereof, many alternatives, modifications and variations will be apparent to those skilled in the art. Accordingly, it is intended to embrace all such alternatives, modifications and variations that fall within the spirit and broad scope of the appended claims. Unless defined otherwise, all technical and scientific terms used herein have the same meaning as commonly understood by one of ordinary skill in the art to which this invention belongs.

1. A method of treatment of cardiac arrhythmias comprising combining a sodium channel blocking medication and a potassium channel blocking medication into a therapeutic dosage and delivering the therapeutic dosage to a patient in treatment of a heart rhythm problem.

2. The method of claim 1 wherein the therapeutic dosage is at least one tablet.

3. The method of claim 1 wherein the heart rhythm problem is atrial fibrillation.

4. The method of claim 1 where the heart rhythm problem is a rhythm problem of a heart chamber other than the left atrium.

5. The method of claim 4 wherein the heart rhythm problem is ventricular tachycardia, supraventricular tachycardia such as atrial flutter, atrial tachycardia or isolated premature atrial or ventricular beats.

6. The method of claim 1 wherein the sodium and potassium channel blocking medications each have a concentration, the concentration of each medication being determined based upon the cardiac arrhythmia being treated, underlying heart rate, QT interval and other comorbid conditions of the patient.

7. The method of claim 6 wherein the sodium channel blocking medication is no less than about 50 milligrams and the potassium channel blocking medication is no greater than about 400 milligrams.

8. The method of claim 7 wherein the sodium channel blocking medication is no greater than about 400 milligrams and the potassium channel blocking medication is no less than about 80 milligrams.

9. The method of claim 1 further comprising a beta blocking medication being combined into the therapeutic dosage.

10. A pharmaceutical composition for the therapeutic treatment of a cardiac arrhythmia in a patient comprising a combination of a sodium channel blocking medication and a potassium channel blocking medication.

11. The pharmaceutical composition of claim 10 wherein the combination is at least one tablet.

12. The pharmaceutical composition of claim 10 wherein the combination is for treatment of atrial fibrillation.

13. The pharmaceutical composition of claim 10 wherein the combination is for treatment of a rhythm problem of a heart chamber other than the left atrium.

14. The pharmaceutical composition of claim 10 wherein the sodium and potassium channel blocking medications each have a concentration, the concentration of each medication being determined based upon the cardiac arrhythmia being treated, underlying heart rate, QT interval and other comorbid conditions of the patient.

15. The pharmaceutical composition of claim 14 wherein the sodium channel blocking medication is within a range of about 50 milligrams to about 400 milligrams.

16. The pharmaceutical composition of claim 14 wherein the potassium channel blocking medication is within a range of about 80 milligrams to about 400 milligrams.

17. The pharmaceutical composition of claim 10 further comprising a beta blocking medication.
